(19) United States
(12) Patent Application Publication
(10) Pub. No.: US 2022/0226066 A1
(43) Pub. Date: Jul. 21, 2022

(54) Formulation for Gastrointestinal Marking

(71) Applicant: Micro-Tech Endoscopy USA Inc., Ann Arbor, MI (US)

(72) Inventor: Scott Haack, Chardon, OH (US)

(21) Appl. No.: 17/579,666

(22) Filed: Jan. 20, 2022

Related U.S. Application Data
(60) Provisional application No. 63/286,311, filed on Dec. 6, 2021.
(60) Provisional application No. 63/140,072, filed on Jan. 21, 2021.

(51) Int. Cl. A61B 90/00; A61M 37/00

(52) U.S. Cl. CPC A61B 90/39; A61M 37/0084; A61B 2090/3966; A61B 2090/3933; A61B 2090/3987; A61B 2090/3904

(57) ABSTRACT

An ink formulation for gastrointestinal tattooing includes water, carbon black, and hyaluronic acid.

### CROSS-REFERENCE TO RELATED APPLICATION(S)

This application claims the benefit of U.S. Provisional Application No. 63/140,072, filed on Jan. 21, 2021, and U.S. Provisional Application No. 63/286,311, filed on Dec. 6, 2021, the contents of which are hereby incorporated by reference in their entirety for all purposes.

### TECHNICAL FIELD

This disclosure relates to gastrointestinal endoscopy.

### BACKGROUND

Tattooing may be used in gastrointestinal endoscopic procedures to visually mark tissue, including lesions for endoscopic resection and resection sites for later examination. Tattooing generally includes injection of an ink formulation, which includes carbon black, into gastrointestinal tissue, such as the submucosa. Conventional ink formulations are, however, subject to migration of the carbon black.

### SUMMARY

Disclosed herein are implementations of an ink formulation tattooing gastrointestinal tissue and for tattooing gastrointestinal tissue.

In implementation, an ink formulation for gastrointestinal tattooing includes water, carbon black, and hyaluronic acid.

The hyaluronic acid may have a molecular weight of 80 kDa, 100 kDa, or more. The hyaluronic acid may be of between 0.2 and 5 times, such as between 0.5 and 2 times, the mass of the carbon black. The hyaluronic acid may be of 0.1% to 2% by mass, such as 0.2% to 0.6% by mass. The water may be of 70%, 75% or more by mass, such as approximately 79.5% by mass. The carbon black may be of 0.1% to 2% by mass, or 0.1% to 1% by mass, such as approximately 0.3% by mass. The ink formulation may further include glycerol, polysorbate (e.g., sorbitan mono-9-octadecenoate poly(oxy-1,2-ethanediyl), also known as polysorbate 80 or by the trade name of Tween 80), and benzyl alcohol. The glycerol may be of 10% to 20% by mass, such as approximately 18% by mass. The polysorbate may be of 0.5% to 1.5% by mass, such as approximately 0.8% by mass. The benzyl alcohol may be of 0.5% to 1.5% by mass, such as approximately 1% by mass. The ink formulation may be sterilized. The ink formulation may not include a dye.

In an implementation, a syringe includes the ink formulation as described above.

In an implementation, a method is provided for tattooing gastrointestinal tissue, which includes injecting the ink formulation as described above.

In an implementation, a radiopaque ink formulation is provided for gastrointestinal imaging, which includes water, carbon black, hyaluronic acid, and a radiopaque formulation.

In an implementation, A method of marking a gastrointestinal site includes providing a first volume of a first formulation in a first syringe and providing a second volume of a second formulation in a second syringe. The method further includes coupling the first syringe and the second syringe with an injection needle via a stopcock, directing a portion of the first volume into the injection needle, and inserting the injection needle into a target site. The method also includes directing a portion of the second volume into the injection needle, and directing another portion of the first volume into the injection needle.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is picture illustrating migration of a conventional ink formulation and a preferred ink formulation 30 days after injection thereof.

FIG. 2 is an x-ray image of another preferred ink formulation after injection thereof.

FIG. 3 is a picture of a device to mix and deliver the preferred ink formulations, according to one embodiment.

FIG. 4 is a picture of another device to mix and deliver the preferred ink formulations, according to another embodiment.

FIG. 5 is a picture of another device to mix and deliver the preferred ink formulations, according to another embodiment.

FIG. 6 is a block diagram of a method of mixing and delivering the preferred ink formulations.

### DETAILED DESCRIPTION

Disclosed herein are embodiments of various formulations for marking tissue in gastrointestinal endoscopic procedures, which may be less susceptible to migration than conventional ink formulations and may also be visible under x-ray. The formulations can include one or more of an ink formulation, a radiopaque ink formulation, and a radiopaque formulation.

As described in further detail below, the formulations generally includes water and hyaluronic acid, and may further include one or more additional materials, such as ink, radiopaque materials, glycerol, benzyl alcohol, and polysorbate. The formulations may be a suspension that includes the water and hyaluronic acid, and which may further include the one or more additional materials.

The ink formulation may, for example, be provided in volumes of five cubic centimeters (cc), ten cc, or fifteen cc in a syringe. For example, it may be advantageous to include ten cc syringes of the ink formulation to provide additional volume of the ink formulation over the five cc syringe for priming needles and related equipment for administering the ink formulation.

The ink formulation includes the water in a concentration of greater than 70% by mass, such as greater than 75% by mass (e.g., 75% to 90% by mass, for example, 75% to 85%, such as approximately 79.5% or approximately 84%). For example, a 10 cc syringe or other container of the ink formulation may include greater than 7.5 grams of water (e.g., 7.5 to 9.0 grams, for example, 7.5 to 8.5 grams, such as approximately 7.95 grams or approximately 8.4 grams).

The ink formulation includes the ink, which may, for example, include carbon black. The ink formulation includes the carbon black in a concentration of 0.005% to 2% by mass (e.g., 0.01% to 1%, such as approximately 0.3% or approximately 1%).

The ink formulation may include the visual marking materials that consist essentially of the carbon black. For example, the ink formulation may not include other ingredients used as visual markers in gastrointestinal endoscopy, such as dyes (e.g., indigo carmine (i.e., 5,5′-indigodisulfonic acid sodium salt) or methylene blue (i.e., methylthioninium chloride)).

The ink formulation includes the hyaluronic acid and, in particular, includes hyaluronic acid having a molecular weight of approximately 80 kDa or greater, for example, preferably 80 kDa to 1,000 kDa (e.g., 80 kDa to 200 kDa, such as approximately 100 kDa).

Without being held to a particular theory, it is believed that hyaluronic acid of such molecular weight may help prevent or hinder migration of the visual marker (i.e., the ink or carbon black) over time, as discussed in further detail below with respect to FIG. 1. Hyaluronic acid of lower and higher molecular weight may still prevent or hinder migration of the ink, albeit possibly with lesser effectiveness. Hyaluronic acid of such molecular weight may also have anti-inflammatory effect, while hyaluronic acid of lower molecular weight may cause inflammation.

Preventing or otherwise hindering migration of the ink (e.g., the carbon black) provides a benefit of maintaining the location of the visual marker for later reference, for example, to support procedures and/or diagnoses to be performed at a later time and/or by another care provider. For example, the visual marker may be a reference for later-performed endoluminal procedures, such as endoscopic submucosal dissection, or open procedures.

Furthermore, preventing or otherwise hindering migration of the ink with the hyaluronic acid and/or the anti-inflammatory effect of the hyaluronic acid may provide other benefits of preventing or otherwise hindering inflammation, fibrosis, and/or granuloma formation that might otherwise occur with the presence and/or spread of ink without the hyaluronic acid.

The ink formulation may further include the hyaluronic acid in a concentration of 0.1% to 2% by mass, such as 0.2% to 1% (e.g., 0.2% to 0.6%, such as approximately 0.4%).

Instead or additionally, the quantity or concentration of the hyaluronic acid may be expressed in terms of relative concentration, quantity, or mass of the ink (i.e., the carbon black). For example, the ink formulation may include the hyaluronic acid at 0.2 to 5 times (e.g., 0.4 to 2 times, such as approximately 0.4 times or approximately 1.3 times) the concentration or mass of the ink (i.e., the carbon black).

As referenced above, the ink formulation may include other ingredients, which may include one or more bulking agents, and/or benzyl alcohol.

The ink formulation may also include one or more bulking agents. The one or more bulking agents may, for example, include glycerol, polysorbate, collagen, and/or methylcellulose. The ink formulation may include the one or more bulking agents, if provided, in a total concentration of 10% to 30% by mass (e.g., 10% to 25%, such as approximately 19%). In embodiments in which the one or more bulking agents include glycerol, the ink formulation may include the glycerol in a concentration of 10% to 25% (e.g., 10% to 20%, such as approximately 18%). In embodiments in which the one or more bulking agents include polysorbate, the ink formulation may include the polysorbate in a concentration of 0.2% to 2% (e.g., 0.5% to 1.5%, such as approximately 0.8%).

In embodiments in which the one or more bulking agents include both glycerol and collagen, the ink formulation may include the glycerol in a concentration of 1% to 10% (e.g., 2% to 5%, such as approximately 3%) and the collagen in a concentration of 5% to 20% by mass (e.g., 10% to 15%, such as approximately 12%).

The ink formulation may also include benzyl alcohol. The ink formulation may include the benzyl alcohol in a concentration of 0.2% to 2% by mass (e.g., 0.5% to 1.5%, such as approximately 1%).

The ink formulation may include methocellulose and/or simethicone instead of or in addition to those various other ingredients described above.

As referenced above, the ink formulation may be a suspension. For example, the ink formulation may be prepared by mixing the ingredients with a centrifuge, which include the water, the ink, and the hyaluronic acid, and may further include one or more of the additional ingredients if provided (e.g., glycerol, polysorbate, collagen, and/or benzyl alcohol).

Furthermore, the ink formulation is sterilized. For example, after mixing the ingredients with the centrifuge to form a pre-sterilization ink formulation, the pre-sterilization ink formulation may be sterilized according to a sterilization process. The sterilization process may include subjecting the pre-sterilization ink formulation to heat and/or pressure for an extended duration to form the ink formulation (e.g., the sterilized ink formulation). For example, the sterilization process may include inserting the pre-sterilization ink formulation into an autoclave and heated to between 100 degrees Celsius and 130 degrees Celsius (e.g., between 110 degrees Celsius and 120 degrees Celsius) for a duration of between 10 minutes and 30 minutes (e.g., approximately 20 minutes), or other sufficient temperate and time duration to sterilize the pre-sterilization ink formulation to form the ink formulation (i.e., the sterilized ink formulation).

It should be noted that the sterilization process may alter the concentrations and/or properties of the materials of the pre-sterilization ink formulation. For example, the sterilization process may reduce the concentration of the water of the pre-sterilization ink formulation (e.g., causing evaporation thereof), thus requiring the pre-sterilization ink formulation to include a higher concentration of water than desired for the final concentration of water in the sterilized ink formulation. Furthermore, the sterilization process may change the molecular weight of the hyaluronic acid, for example, reducing the molecular weight of the hyaluronic acid, thus requiring that the pre-sterilization ink formulation include hyaluronic acid of a higher molecular weight that desired for the final molecular weight of the hyaluronic acid in the sterilized ink formulation.

In a first preferred example, the ink formulation includes by mass 84% water, 1% carbon black, 0.4% hyaluronic acid having a molecular weight of 100 kDa, 3% glycerol, and 11.6% collagen.

In a second preferred example, the ink formulation includes by mass 79.5% water, 0.3% carbon black, 0.4% hyaluronic acid, 18% glycerol, 0.8% polysorbate, and 1% benzyl alcohol. The hyaluronic acid has a molecular weight of approximately 100 kDa, which was provided in the pre-sterilization ink formulation with a molecular weight of approximately 1,000 kDa.

Referring to FIG. 1, testing was performed in which a conventional ink formulation 102 and the second preferred ink formulation 104 were both injected to tattoo (i.e., provide markers) in a colon 100 of a subject. As shown in FIG. 1, 30 days after injection, the conventional ink formulation 102 (outlined in dash-dash lines) migrated significantly to several times its original size (e.g., more than twenty times the visible area), while the second preferred ink formulation 104 (outlines in dash-dot lines) migrated substantially less (e.g., less than five times the original visible area).

The conventional ink formulation included by mass greater than 76% water, 10% to 20% glycerol, 0.5% to 1.5% polysorbate, 0.5% to 1.5% benzyl alcohol, and 0.01% to 1% carbon black. The second preferred ink formulation is as described above.

The radiopaque ink formulation can include the ink formulation as described above and can also include a radiopaque formulation. Accordingly, the radiopaque ink formulation can be viewed with a visible light camera (e.g., a camera inserted into the target site) and under x-ray imaging (e.g., still x-ray images and/or live x-ray images).

The radiopaque formulation may, for example, be provided in volumes of one cc, two ccs, or three ccs in a syringe. For example, it may be advantageous to include three cc syringes of the radiopaque formulation to provide additional volume of the radiopaque formulation over smaller syringes marking multiple locations without changing the syringe being used.

The radiopaque formulation can include any biocompatible material that is visible under x-ray. For example, the radiopaque formulation can include one or more biocompatible metals such as titanium (Ti), tantalum (Ta), stainless steel, platinum (Pt), gold (Au), silver (Ag), and any other biocompatible metal. The radiopaque formulation can also include one or more biocompatible polymers such as poly-ether-ether-ketone (PEEK), polycarbonate, etc., where the biocompatible polymers further include radiopaque material such as barium sulfate or one or more of the biocompatible metals above.

The radiopaque formulation can be provided as a powder that includes a group of individual grains of the radiopaque formulation. In some embodiments, a size (e.g., a diameter for grains that are spherical, or the longest measurement for grains that are not spherical) of the individual grains of the radiopaque formulation are between ten and two-hundred micrometers (e.g., between twenty and one-hundred-eighty micrometers, such as between forty and one-hundred-fifty micrometers).

The radiopaque formulation can also include, for example, hyaluronic acid having a molecular weight of approximately 80 kDa or greater, for example, preferably 80 kDa to 1,000 kDa (e.g., 80 kDa to 200 kDa, such as approximately 100 kDa).

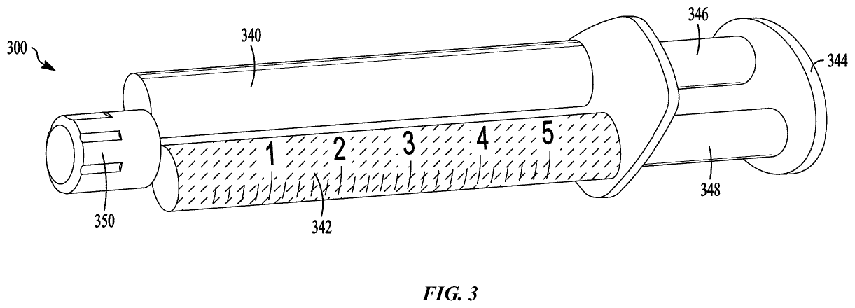

The radiopaque formulation can be mixed, for example, with the ink formulation (e.g., to create a radiopaque ink formulation) prior to being injected into a target site. The radiopaque formulation can be mixed by hand, in a centrifuge (as described above), or by any other known method. In some embodiments, the radiopaque formulation and the ink formulation can be mixed as they are being injected into the target site. The radiopaque formulation and the ink formulation can also be injected into the target site separately. Devices and methods used to inject the radiopaque ink formulation into a target site are further described with reference to FIGS. 3-6.

The radiopaque formulation can also be sterilized in the same manner described above with respect to the ink formulation.

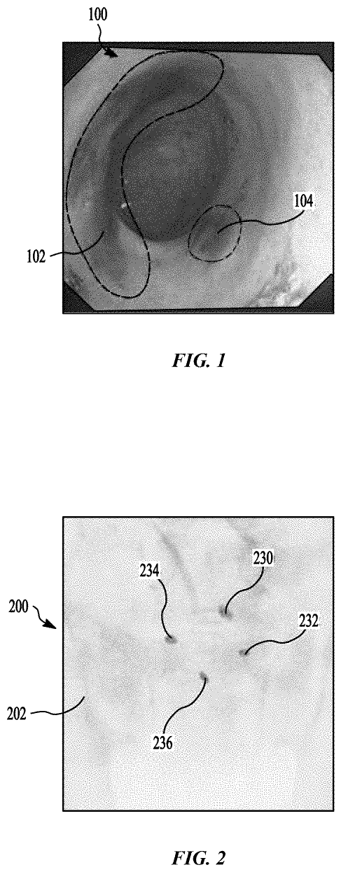

With reference to FIG. 2, an x-ray image 200 of the radiopaque ink formulation after injection thereof in a colon 202 is shown. The x-ray image 200 was taken twenty-eight days after the radiopaque ink formulation was injected in the colon 202. The radiopaque ink formulation was injected at four locations in the colon 202 (e.g., a location 230, a location 232, a location 234, and a location 236). As shown, twenty-eight days after injection each of the locations 230-236 is distinct from each other, allowing for accurate targeting of each of the locations 230-236 under x-ray guidance.

In some embodiments, the radiopaque formulation is provided without other components or constituents. Accordingly, when using only the radiopaque formulation, the radiopaque formulation can be viewed under x-ray imaging (e.g., still x-ray images and/or live x-ray images) but not with a visible light camera.

In some instances, the constituents of the formulations disclosed herein may not remain mixed homogeneously after manufacturing, sterilization, shipping, and any other handling involved. Therefore, it may be necessary to ensure the constituents are properly mixed prior to injection into a target site.

FIG. 3 is a picture of a device 300 to mix and deliver the formulations, according to one embodiment. The device 300 is shown as a double-barreled syringe and includes a first barrel 340, a second barrel 342, a plunger 344, and a tip 350. Each component of the device 300 is generally manufactured from a polymer that does not react with the components held within it. Examples of materials from which the device 300 can be manufactured include polyethylene and polycarbonate.

The first barrel 340 and the second barrel 342 are generally cylindrical and tubular in shape, but any other suitable geometric configurations can be used. The first barrel 340 and the second barrel 342 are separate and distinct and are configured to store components to be mixed. For example, the first barrel 340 can store the ink formulation and the second barrel 342 can store the radiopaque formulation.

The plunger 344 includes a first arm 346 partially disposed within the first barrel 340 and a second arm 348 partially disposed within the second barrel 342. Each of the first arm 346 and the second arm 348 may include a sealing component (e.g., an o-ring, a gasket, etc.) to seal against an inner wall of the first barrel 340 and the second barrel 342, respectively. As the plunger 344 is pressed toward the first barrel 340 and the second barrel 342, the sealing components interact with the inner walls to drive the components stored within the device 300 toward the tip 350.

The tip 350 is shown covered in FIG. 2, however the tip 350 includes a mixing component (not shown) configured to mix the contents of the first barrel 340 with the contents of the second barrel 342 as each of the contents flow through the mixing component. In some embodiments, the mixing component can be a helical mixer that forces the contents to flow through the tip in a helical manner to promote mixing. The mixing component can also include a tortuous path of switchbacks through which the contents must flow to exit the tip.

A needle can also be coupled with the tip 350 such that the contents flow through the needle to be injected into the target site.

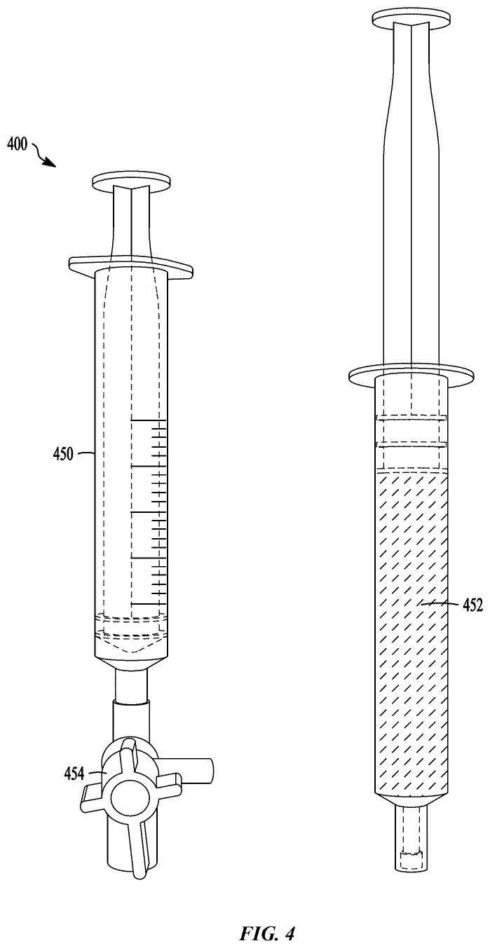

FIG. 4 is a picture of another device 400 to mix and deliver the formulations, according to another embodiment. The device 400 is shown to include a first syringe 450, a second syringe 452, and a stopcock 454. The first syringe 450 and the second syringe 452 are the same size (e.g., both can hold volumes of five cc, ten cc, fifteen cc, etc.). As shown, the first syringe 450 is empty with the plunger fully depressed, and the second syringe 452 includes any one of the formulations disclosed herein.

To mix the constituents of the formulation to create a homogeneous mix or an approximately homogeneous mix, the first syringe 450 and the second syringe 452 are each coupled with the stopcock 454, and the stopcock is positioned to allow flow between the first syringe 450 and the second syringe 452. Mixing is accomplished by fully depressing the plunger of the syringe in which the constituents are stored to direct the constituents to the empty syringe, thereby filling the empty syringe. In the example configuration shown in FIG. 4, the plunger of the second syringe 452 is depressed and the contents of the second syringe (e.g. 452 are directed through the stopcock 454 and into the first syringe 450, causing the plunger of the first syringe to move to receive the contents. This process can be repeated as needed, thereby moving the contents back and forth between the first syringe 450 and the second syringe 452 until a desirable mix is achieved. After achieving the desirable mix of the formulation, the syringe containing the formulation (e.g., either the first syringe 450 or the second syringe 452) is removed from the stopcock 454. A needle can be coupled with a tip of the syringe, inserted at the target site, and the formulation can be delivered to the target site by depressing the plunger.

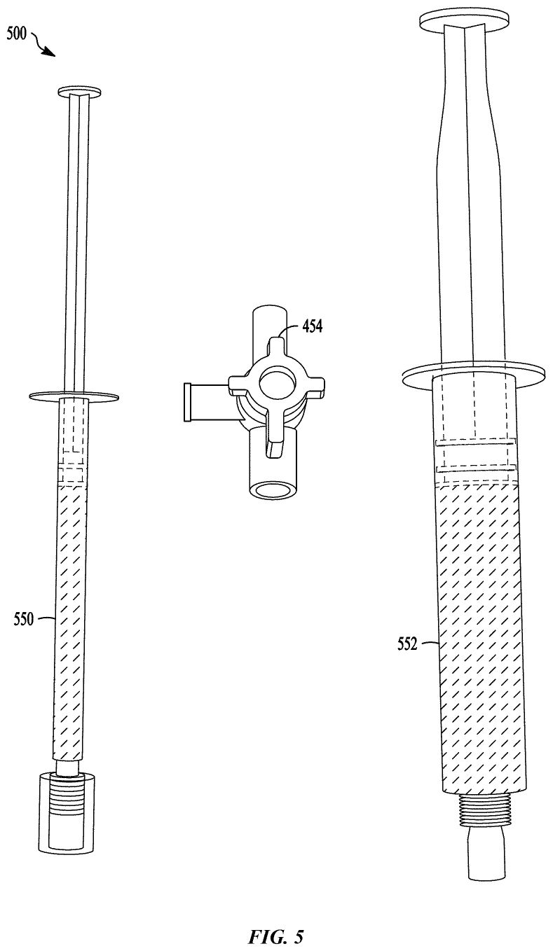

FIG. 5 is a picture of another device 500 to mix and deliver the formulations, according to another embodiment. The device 500 is shown to include a first syringe 550, a second syringe 552, and the stopcock 454. The first syringe 450 and the second syringe 452 are different sizes. For example, the first syringe 550 may be configured to hold a volume of one cc, two ccs, or three ccs. The second syringe 552 may be configured to hold a volume of five ccs, ten ccs, or fifteen ccs. The volumes disclosed are examples, and the syringes can be configured to hold different volumes as long as the first syringe 550 is configured to hold a lower volume than the second syringe 552.

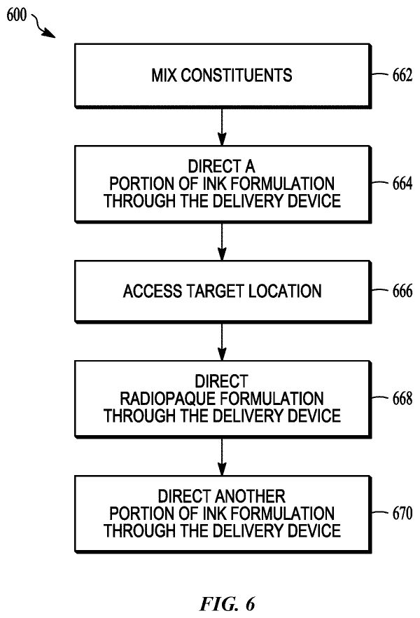

As shown, the first syringe 550 includes the radiopaque formulation, and the second syringe 552 includes the ink formulation. In some embodiments, the second syringe 552 includes all of the constituents of the ink formulation without the ink (e.g., water, hyaluronic acid, and/or bulking agents), referred to herein as the “non-ink formulation”. The non-ink formulation can be mixed and sterilized as described above with respect to the ink formulation. The formulation in the second syringe 552 may be mixed using the technique described with reference to FIG. 4. In some embodiments, the first syringe 550 and the second syringe 552 can be coupled via the stopcock 454 and the contents of first syringe 550 and the second syringe 552 can be mixed using a technique similar to that described with reference to FIG. 4 prior to injecting into the target site. Additionally, the device 500 can be used to inject the contents of the first syringe 550 and the second syringe 552 into the target site without first mixing the contents together. This injection method is further described with reference to FIG. 6.

FIG. 6 is a block diagram of a method 600 of mixing and delivering the formulations. As shown, at operation 662 constituents are mixed. For example, constituents of the ink formulation may be mixed according to the technique described with reference to FIG. 4. In other embodiments, the constituents can be mixed by hand (e.g., hand shaken, etc.) or with any other kind of mixer (e.g., vibration mixer, etc.).

At operation 664, a first amount of the ink formulation can be directed through a delivery device. For example, and with reference to FIG. 5, the first syringe 550 and the second syringe 552 can be coupled with the stopcock 454, and an injection needle (not shown) can also be coupled with the stopcock 454. The stopcock 454 can be adjusted to allow flow from the second syringe 552 to the needle, and the plunger of the second syringe 552 can be depressed until the ink formulation exits the tip of the injection needle, thereby priming the injection needle.

At operation 666, the target location is accessed. For example, the location to be marked is observed using a visible light camera, x-ray imaging, or both. The injection needle (while still coupled with the stopcock 454, the first syringe 550, and the second syringe 552) is inserted to the target location until the tip of the injection needle reaches the target location. The position of the tip of the injection needle can be confirmed via the visible light camera, x-ray imaging, or both.

At operation 668, the radiopaque formulation is directed through the device 500. For example, the stopcock 454 is adjusted to allow flow between the first syringe 550 and the injection needle. The plunger of the first syringe 550 can then be depressed to direct the radiopaque formulation through the device 500. In some embodiments, the plunger of the first syringe 550 is fully depressed to direct all of the radiopaque formulation through the device 500. In some embodiments, the plunger of the first syringe 550 is depressed only a portion of the length of the first syringe 550 to deliver only a portion of the radiopaque formulation through the device 500.

At operation 670, a second amount of the ink formulation is directed through the device 500. Based on the size of the injection needle, the radiopaque formulation may or may not reach the target location at operation 668. For example, the injection needle may be sized such that it can accommodate a volume of approximately one cc and the volume of radiopaque formulation injected may be less than one cc. To ensure that the entire volume of radiopaque formulation reaches the target site, a second amount of the ink formulation is directed through the device 500.

For example, the stopcock 454 is adjusted to allow flow between the second syringe 552 and the injection needle. The plunger of the second syringe 552 is then depressed to direct the second amount of the ink formulation through the device 500. In some embodiments, the second amount of the ink formulation is approximately the same amount as the first amount of the ink formulation. The second amount of the ink formulation can also be greater than or less than the first amount of the ink formulation as long as the second amount of the ink formulation directs the entire amount of the radiopaque formulation into the target site.

The method 600 is described above with the second syringe 552 including the ink formulation such that, after injection, the mark can be viewed with both a visible light camera and under x-ray. The second syringe 552 can also include the non-ink formulation such that the mark can be viewed only under x-ray.

The term “approximately,” as used herein, includes a range of +/− 20% of the indicated value.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. An ink formulation for gastrointestinal tattooing comprising:
water;
carbon black; and
hyaluronic acid.

2. The ink formulation according to claim 1, wherein the hyaluronic acid has a molecular weight of 80 kDa or more.

3. The ink formulation according to claim 2, wherein the hyaluronic acid has a molecular weight of 100 kDa or more.

4. The ink formulation according to claim 1, comprising the hyaluronic acid of 0.1% to 2% by mass.

5. The ink formulation according to claim 4, comprising the hyaluronic acid of 0.2% to 0.6% by mass.

6. The ink formulation according to claim 1, wherein the hyaluronic acid is of a concentration by mass of between 0.2 and 5 times that of the carbon black.

7. The ink formulation according to claim 6, wherein the concentration by mass of the hyaluronic acid is between 0.5 and 2 times that of the carbon black.

8. The ink formulation according to claim 1, comprising the water of 70% or more by mass and the carbon black of 0.01% to 2% by mass.

9. The ink formulation according to claim 8, comprising the water of 75% or more by mass and the carbon black of 0.1% to 1% by mass.

10. The ink formulation according to claim 9, comprising the water of approximately 79.5% by mass and the carbon black of approximately 0.3% by mass.

11. The ink formulation according to claim 1, further comprising glycerol, polysorbate, and benzyl alcohol.

12. The ink formulation according to claim 11, comprising the glycerol of 10% to 20% by mass, the polysorbate of 0.5% to 1.5% by mass, and the benzyl alcohol of 0.5% to 1.5% by mass.

13. The ink formulation according to claim 12, comprising the glycerol of approximately 18% by mass, the polysorbate of approximately 0.8% by mass, and the benzyl alcohol of approximately 1% by mass.

14. The ink formulation according to claim 1, wherein the ink formulation is sterilized.

15. The ink formulation according to claim 1, wherein the ink formulation does not include a dye.

16. The ink formulation according to claim 1, further comprising a bulking agent.

17. A syringe comprising the ink formulation according to claim 1.

18. A method of tattooing gastrointestinal tissue, comprising injecting the ink formulation according to claim 1 into gastrointestinal tissue.

19. A radiopaque ink formulation for gastrointestinal imaging, comprising:
water;
carbon black;
hyaluronic acid; and
a radiopaque component.

20. The radiopaque ink formulation of claim 19, wherein the radiopaque component includes a biocompatible metal.

21. The radiopaque ink formulation of claim 19, wherein the radiopaque component includes a biocompatible polymer.

22. The radiopaque ink formulation of claim 19, further comprising a bulking agent.

23. A method of marking a gastrointestinal site, comprising:
providing a first volume of a first formulation in a first syringe;
providing a second volume of a second formulation in a second syringe;
coupling the first syringe and the second syringe with an injection needle via a stopcock;
directing a portion of the first volume into the injection needle;
inserting the injection needle into a target site;
directing a portion of the second volume into the injection needle; and
directing another portion of the first volume into the injection needle.

24. A radiopaque formulation for gastrointestinal imaging, comprising:
water;
hyaluronic acid; and
a radiopaque component.

25. The radiopaque formulation of claim 24, further comprising a bulking agent.
